(19) United States
(12) Patent Application Publication
(10) Pub. No.: US 2013/0274126 A1
(43) Pub. Date: Oct. 17, 2013

(54) SENSOR CHIP AND MEASUREMENT METHOD USING THE SAME

(71) Applicant: HITACHI, LTD., Tokyo (JP)

(72) Inventors: Yu ISHIGE, Tokyo (JP); Masao KAMAHORI, Tokyo (JP)

(73) Assignee: HITACHI, LTD., Tokyo (JP)

(21) Appl. No.: 13/802,970

(22) Filed: Mar. 14, 2013

(30) Foreign Application Priority Data
Apr. 17, 2012 (JP) 2012-093824

(51) Int. Cl. C12Q 1/04

(52) U.S. Cl. CPC C12Q 1/04
USPC 506/9; 506/11; 506/18

(57) ABSTRACT

Provided is a device that can detect cells or bacteria in units of a single cell or bacterium, and can further measure the amounts of activity of cells or bacteria or responses of the cells or bacteria to drugs in units of a single cell or bacterium. A plurality of partitioned regions each having about the same size as a cell or a bacterium is provided, and a plurality of types of electrical sensors 201 and 202 are arranged in each partitioned region.

### CLAIM OF PRIORITY

The present application claims priority from Japanese patent application JP 2012-093824 filed on Apr. 17, 2012, the content of which is hereby incorporated by reference into this application.

### BACKGROUND

1. Technical Field

The present invention relates to a sensor chip capable of measuring microorganisms and living substances with high accuracy and high sensitivity through electrical measurement, and a measurement method using the sensor chip.

2. Background Art

A biosensor is a sensor formed by combining biomolecules such as antibodies or enzymes with mainly an electrochemical sensor and thus has high selectivity of antibodies or enzymes. For example, when glucose oxidase, which acts on glucose to selectively react with oxygen, is combined with a sensor that electrochemically measures oxygen, it is possible to produce a glucose sensor (see Non-Patent Document 1). With respect to biosensors in the early days, a sensing portion such as an electrode and a measuring portion that performs electrical measurement have been connected with a wire. Then, a semiconductor fabrication technology has been introduced to fabricate a chip with an integrated structure of a sensing portion and a measuring portion, and thus achieve a reduction in size and an increase in sensitivity of the sensor. For example, when an ion sensitive field effect transistor (ISFET) with pH sensitivity is combined with urease that catalyzes the hydrolysis of urea into protons, it is possible to produce a urea sensor that can obtain an electrical signal in accordance with the concentration of urea (Non-Patent Document 2).

With the progress of semiconductor fabrication technologies in recent years, it has become possible to fabricate a sensor array in which a plurality of sensors are mounted on a single chip. Such a sensor array is also applied to a biosensor. For example, a sensor array is applied to measurement of a plurality of cells (Non-Patent Document 3) or measurement of a distribution of potentials in neurons that are nerve cells (Patent Document 1, Non-Patent Document 4). Further, when a sensor array with electrodes each having about the same size as a bacterium or a virus to be measured is used, it is possible to measure the target to be measured from a change in the electrical characteristics of each electrode (Patent Document 2). For such a biosensor array, a sensor array having only one type of sensors has been used.

### PATENT DOCUMENTS

Patent Document 1: JP Patent Publication (Kohyo) No. 2003-513274A
Patent Document 2: JP Patent Publication (Kokai) No. 2011-232328A

### NON-PATENT DOCUMENTS

Non-Patent Document 1: S J Updike, G P Hicks, Nature, 1967, 214, 986
Non-Patent Document 2: Miyahara, Y., Moriizumi, T., Sens. Acutuators, 1985, 7, 1-10
Non-Patent Document 3: M Jenkner et al, IEEE Journal of Solid-State Circuits, 2004, 39, 2431
Non-Patent Document 4: F Heer et al, IEEE Journal of Solid-State Circuits, 2006, 41, 1620

### SUMMARY

When a sensor array with electrodes each having about the same size as a bacterium or a virus to be measured is used to measure the target to be measured (Patent Document 2), it is possible to obtain information about the size of the target to be measured as well as the selectivity of antibodies immobilized on the electrodes. Meanwhile, although the amount of activity of a bacterium, a response of a bacterium to drugs, and the like are information that are useful to identify the bacterium, such information has been lacking.

As another application of a sensor array, a well is formed around each electrode on a sensor chip, and a bead having an enzyme or an antibody immobilized thereon is arranged in each well, and then an enzyme reaction or an antigen-antibody reaction occurring in each well is concurrently detected with each sensor. At this time, arranging different types of beads (which are modified by different enzymes or antibodies) in the respective wells has been quite complex and difficult in terms of production.

A representative configuration of the present invention is a sensor chip having a board, a plurality of partitioned regions provided on a surface of the board, a plurality of types of sensing portions provided in each partitioned region, and detection portions connected to the respective sensing portions. When a target to be measured is a cell or a bacterium, each partitioned region is designed to have about the same size as the target to be measured. When beads having enzymes or antibodies immobilized thereon are used as a measuring tool, each well is formed such that a single bead is arranged in each partitioned region, and is surrounded by a wall that is higher than the diameter of the bead so that the bead that has once entered the well will not fall out.

One of the plurality of sensing portions is a sensing portion for detecting the presence or absence of a cell or a bacterium to be measured or a bead in each partitioned region, and the other sensing portion is a sensing portion for measuring a metabolite generated by the target to be measured or a reaction product of the enzyme.

A measurement method according to one embodiment of the present invention includes: introducing a solution containing a target to be measured onto a sensor chip, the sensor chip having a board and a plurality of partitioned regions provided on a surface of the board, and each partitioned region having a first sensing portion for detecting the presence or absence of the target to be measured and a second sensing portion for measuring a metabolite of a substrate; detecting, with the first sensing portion, the presence or absence of the target to be measured in each partitioned region; introducing a substrate onto the sensor chip; and measuring, with the second sensing portion, a metabolite of the substrate generated by the target to be measured.

A measurement method according to another embodiment of the present invention includes: introducing a solution containing first beads having first enzymes immobilized thereon onto a sensor chip, the sensor chip having a board and a plurality of wells formed on a surface of the board, and each well having a first sensing portion for detecting the presence or absence of the bead and a second sensing portion for measuring a metabolite of a substrate; detecting, with the first sensing portion of each well, the presence or absence of one of the first beads; removing excess first beads that have not entered the well; storing a well in which the first bead is detected and the first enzyme in association with each other; introducing a solution containing second beads having second enzymes immobilized thereon onto the sensor chip; removing excess second beads that have not entered the well; detecting, with the first sensing portion, the presence or absence of one of the second beads in each well excluding the well in which the first bead is detected; storing the well in which the second bead is detected and the second enzyme in association with each other; introducing a mixture of an analyte and a substrate onto the sensor chip; measuring, with the second sensing portion, a reaction product of the first enzyme or the second enzyme; and obtaining information about a measurement item measured with the second sensing portion with reference to the stored correspondence between the well and the first enzyme or the second enzyme.

A measurement method according to still another embodiment of the present invention includes: introducing a solution containing first beads having first enzymes immobilized thereon onto a sensor chip, the sensor chip having a board and a plurality of wells formed on a surface of the board, and each well having a first sensing portion for detecting the presence or absence of the bead and a second sensing portion for measuring a metabolite of a substrate; detecting, with the first sensing portion of each well, the presence or absence of one of the first beads; removing excess first beads that have not entered the well; storing a well in which the first bead is detected and the first enzyme in association with each other; introducing a solution containing second beads having second enzymes immobilized thereon onto the sensor chip; removing excess second beads that have not entered the well; detecting, with the first sensing portion, the presence or absence of one of the second beads in each well excluding the well in which the first bead is detected; storing the well in which the second bead is detected and the second enzyme in association with each other; introducing an analyte containing a substance to be measured onto the sensor chip, and causing the substance to be measured to be trapped by the first enzyme immobilized on the first bead or the second enzyme immobilized on the second bead; causing the substance to be measured trapped by the first enzyme or the second enzyme to react with an enzyme-labeled secondary antibody; introducing a solution containing a substrate to react with the enzyme labeled on the secondary antibody; measuring, with the second sensing portion, a reaction product of the enzyme; and obtaining information about a measurement item measured with the second sensing portion with reference to the stored correspondence between the stored well and the first antibody or the second antibody.

According to the present invention, a sensor that detects the presence or absence of a target to be measured and a sensor that measures a metabolite of a substrate are arranged such that they co-exist in a range having about the same size as a cell or a bacterium to be measured, whereby it is possible to perform, in addition to the detection of the presence or absence of the cell or the bacterium, measurement of the amount of activity of each bacterium or cell, and thus improve the accuracy of identification of the target to be measured. In addition, a plurality of wells are formed on the board, and a sensor that detects the presence or absence of a bead and a sensor that measures an enzyme reaction product are arranged such that they co-exist in each well, whereby it is possible to recognize which bead is arranged in which well even when beads having enzymes or antibodies immobilized thereon are randomly arranged in the wells, and thus measure a plurality of items concurrently.

Other problems, configurations, and advantages will become apparent from the following description of embodiments.

### BRIEF DESCRIPTION OF THE DRAWINGS

FIG. 1 is a schematic view showing an example of a sensor chip of the present invention;

FIGS. 2A and 2B are a plan schematic view and a cross-sectional schematic view, respectively, of a single partitioned region of an example of a sensor ship;

FIGS. 3A and 3B are a plan schematic view and a cross-sectional schematic view, respectively, of a sensor that is modified by antibodies for trapping a specific bacterium;

FIGS. 4A and 4B are schematic views showing a state in which a bacterium 210 to be measured is trapped by the sensor;

FIGS. 5A and 5B are a cross-sectional schematic view and a plan schematic view, respectively, of an example of a sensor that has a doughnut-shaped electrode and in which a portion surrounded by the electrode is modified by antibodies;

FIGS. 6A and 6B are a cross-sectional schematic view and a plan schematic view, respectively, of a state in which a bacterium binds to the antibodies that modify the sensor shown in FIG. 5;

FIG. 7 is a schematic view showing the arrangement for impedance measurement;

FIGS. 8A and 8B are charts showing an impedance spectrum before the binding of a bacterium and an impedance spectrum after the binding of a bacterium, respectively;

FIGS. 9A and 9B are a plan schematic view and a cross-sectional schematic view, respectively, of a single partitioned region of an example of a sensor ship;

FIGS. 10A and 10B are a plan schematic view and a cross-sectional schematic view, respectively, of a single partitioned region of an example of a sensor ship;

FIG. 11 is a schematic view showing an example of an enzyme sensor array;

FIGS. 12A and 12B are a plan schematic view and a cross-sectional schematic view, respectively, of a single partitioned region of an example of an enzyme sensor array;

FIG. 13 is a schematic view showing a state in which an enzyme-immobilized bead is arranged in a single partitioned region of an enzyme sensor array;

FIGS. 14A and 14B are a plan schematic view and a cross-sectional schematic view, respectively, showing an another example of a single partitioned region of an enzyme sensor array;

FIG. 15 is a schematic view of a measurement chip;

FIG. 16 is an illustration diagram of the procedures for arranging an enzyme-immobilized bead in each well of a sensor chip;

FIG. 17 is a diagram showing the correspondence between the positions of wells on a sensor chip and the types of enzyme-immobilized beads arranged in the respective wells; and

FIG. 18 is a schematic view of a measuring device on which a sensor chip is set.

### DETAILED DESCRIPTION

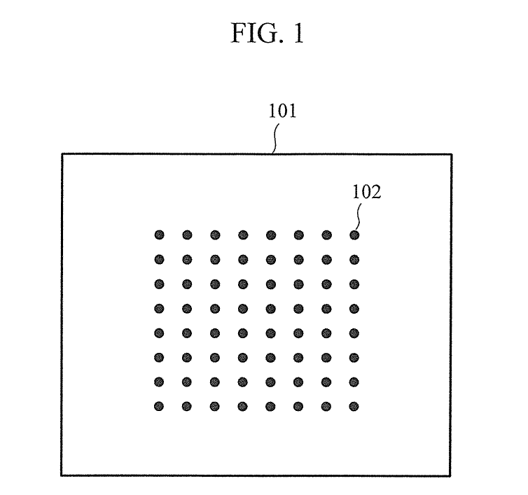

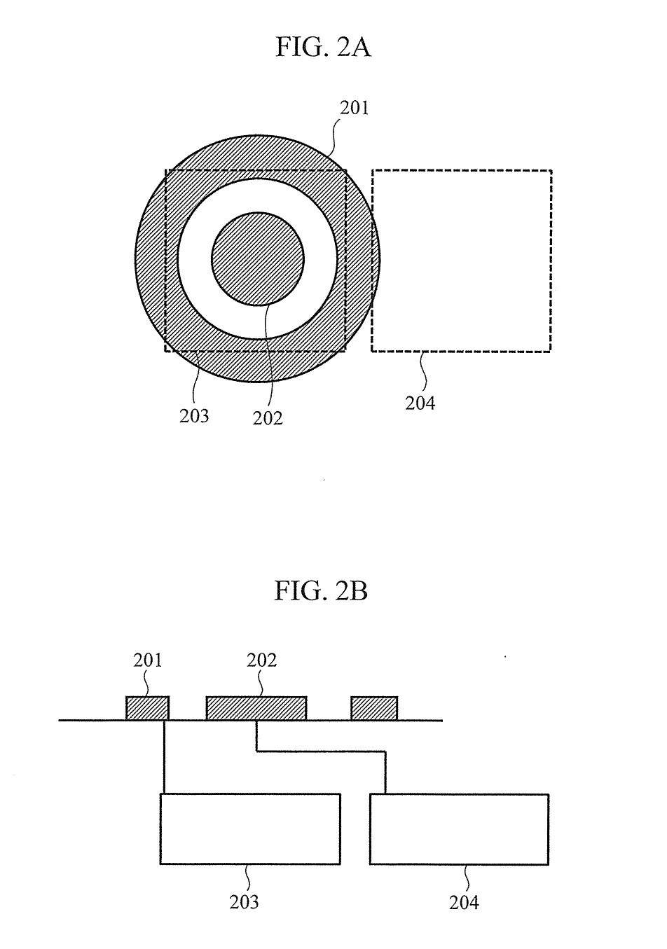

Hereinafter, embodiments of the present invention will be described with reference to the drawings. FIG. 1 is a schematic view showing an example of a sensor chip 101 in which a sensor that detects the presence or absence of a bacterium and a sensor that measures metabolites of a substrate co-exist in a single partitioned region 102 (indicated by a solid black circle), and a plurality of such partitioned regions are arranged in a two-dimensional array. FIGS. 2A and 2B are schematic views showing a single partitioned region. FIG. 2A is a plan schematic view and FIG. 2B is a cross-sectional view. A circular sensing portion 202 of the sensor that measures metabolites of a substrate is arranged such that it is surrounded by a doughnut-shaped sensing portion 201 of the sensor that detects the presence or absence of a bacterium. The sensing portions 201 and 202 are connected to measuring portions 203 and 204, respectively, in the sensor chip via wires. When the measuring portion 203 is a measuring portion for alternating-current impedance, the wire between the sensing portion 201 and the measuring portion 203 is desirably short as shown in FIG. 2. This is because using a long wire for measuring alternating-current impedance could increase the parasitic capacitance, which in turn could lower the measurement sensitivity. Consequently, as shown in FIG. 2, the wire between the sensing portion 201 and the measuring portion 203 is shorter than the wire between the sensing portion 202 and the measuring portion 204. This is because while the sensing portion 201 is required to have a size of about several μm, which is about the same size as a bacterium, the measuring portions 203 and 204 typically need larger areas than that.

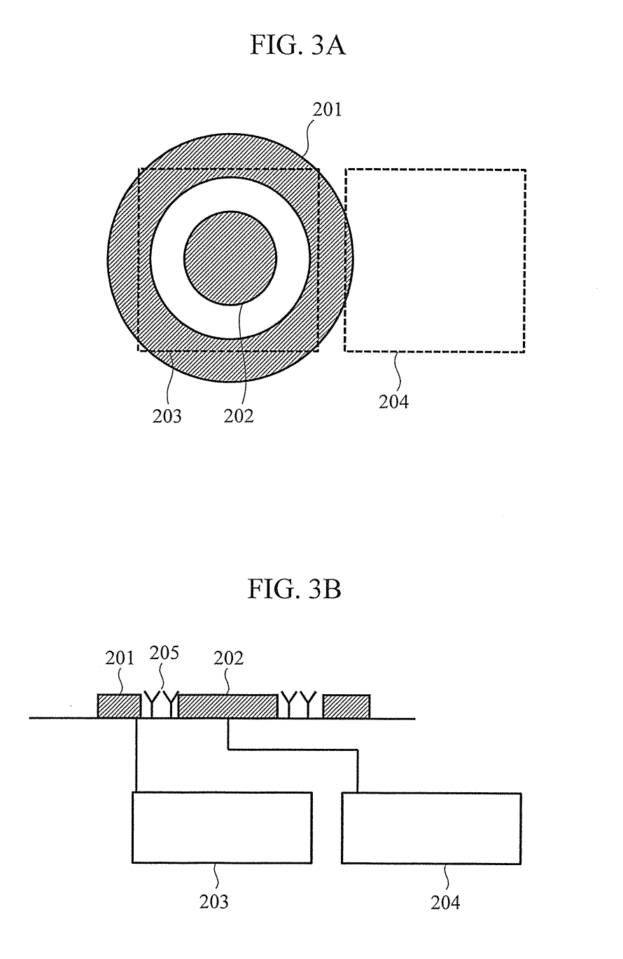

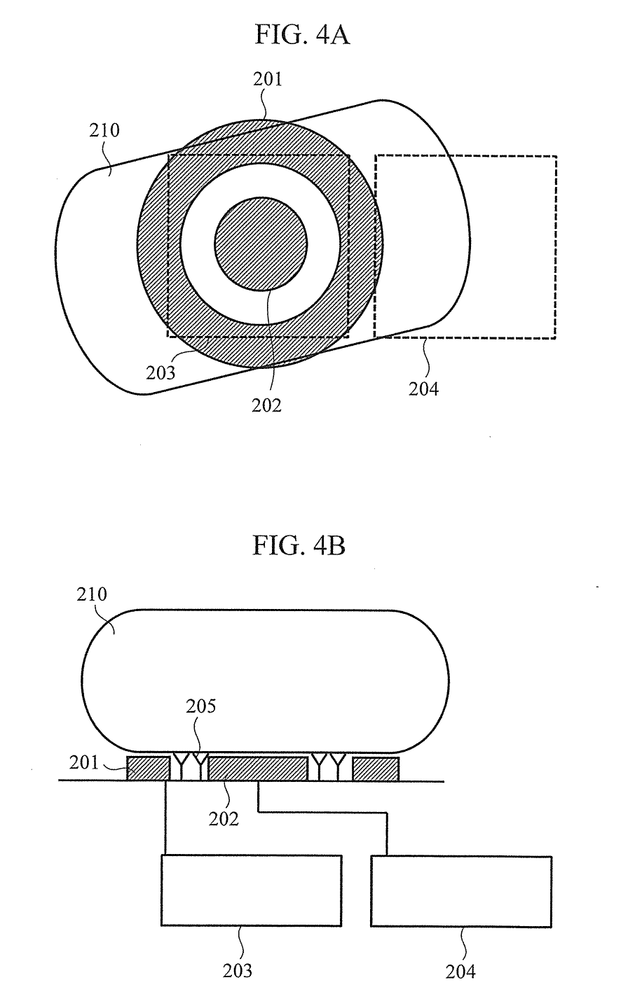

FIGS. 3A and 3B are schematic views of a case where the sensor shown in FIGS. 1, 2A, and 2B is modified by anti-bodies 205 for trapping a specific bacterium. In order to provide selectivity with respect to a specific bacterium, a region between the doughnut-shaped electrode and the circular electrode is modified by antibodies 205 against the specific bacterium. FIGS. 4A and 4B are schematic views showing a state in which a bacterium 210 to be measured is trapped by the sensor shown in FIGS. 3A and 3B. As a region around the surface of the sensing portion 201 is covered with the trapped bacterium 210, a signal output from the measuring portion 203 connected to the sensing portion 201 will change. Through the signal change, trapping of the bacterium can be detected. Herein, when a substrate to be metabolized by the bacterium such as glucose is introduced, the substrate is metabolized by the bacterium, releasing protons and the like as a product. Release of the product is detected as a change in the intensity of a signal output from the measuring portion 204 connected to the sensing portion 202. Meanwhile, when the introduced substrate is not metabolized by the bacterium, the signal intensity does not change. By measuring a response of when various substrates are introduced, it is possible to determine the type of the bacterium as well as whether the bacterium is live or not. This has become possible as the sensing portion 201 that detects the presence or absence of a bacterium and the sensing portion 202 that measures metabolites of a substrate co-exist in a single partitioned region with about the same size as the bacterium.

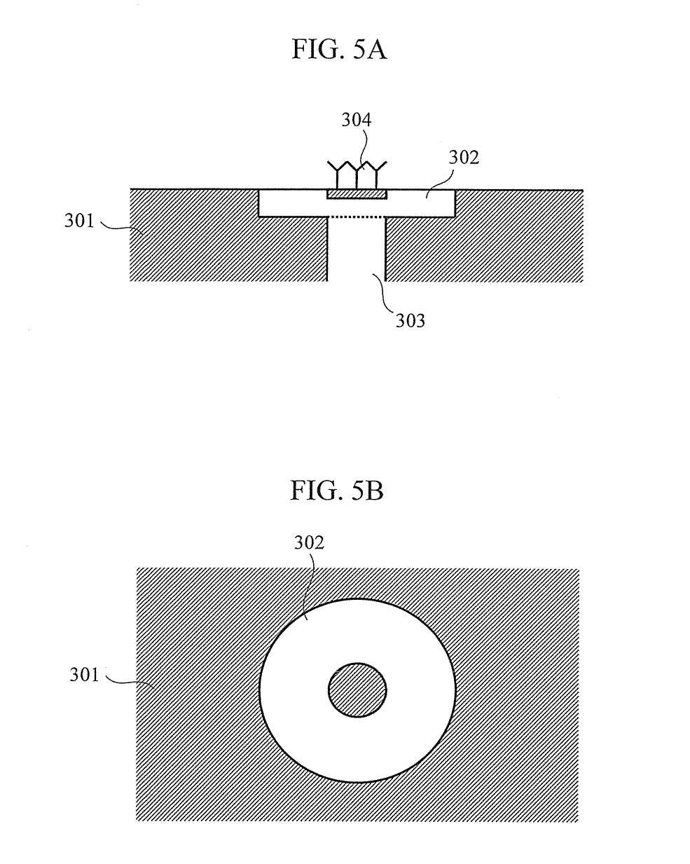

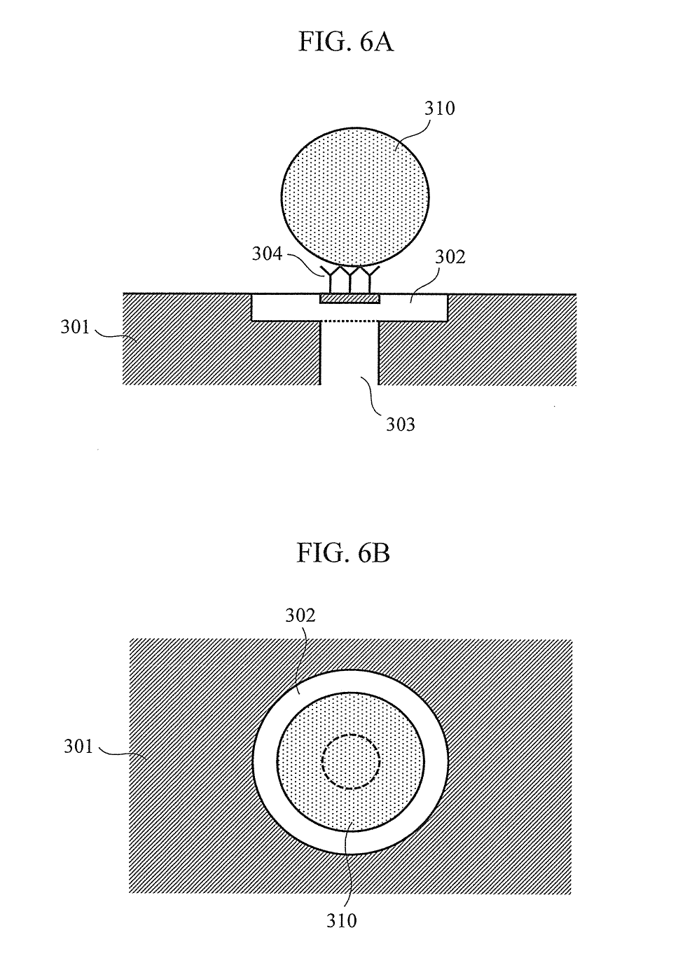

For detecting the presence or absence of a bacterium, alternating-current impedance measurement or direct-current redox current measurement can be used. FIGS. 5A to 8B are illustration diagrams showing exemplary detection of an object on an electrode through alternating-current impedance measurement. FIGS. 5A and 5B show an example of a sensor that has a doughnut-shaped electrode 302 (an outer diameter of 1.5 μm and an inner diameter of 0.8 μm) formed on a board 301 and in which a portion surrounded by the electrode is modified by antibodies 304. The electrode 302 is connected to a measuring portion, which measures alternating-current impedance, via a wire 303. FIGS. 6A and 6B show a state in which a bacterium 310 with a diameter of 1 μm binds to the antibodies 304 that modify the sensor shown in FIGS. 5A and 5B.

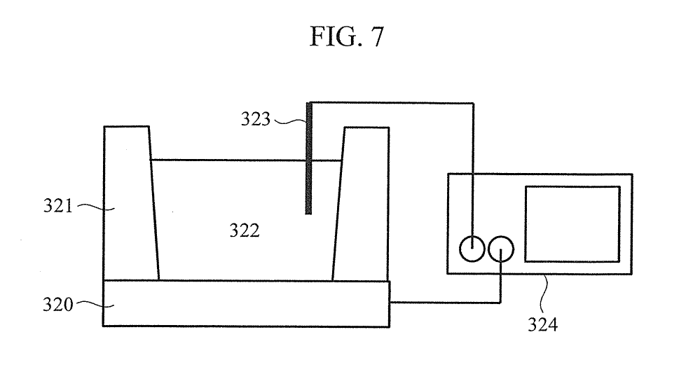

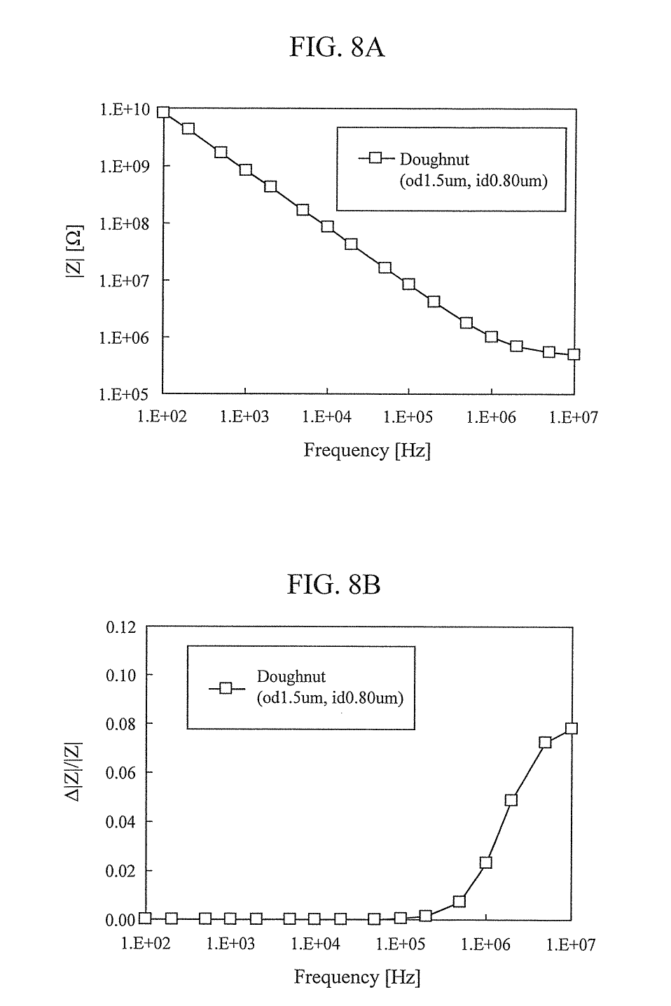

As shown in FIG. 7, a 100 mM sodium sulfate aqueous solution was poured as a sample solution 322 into a cell 321 formed on an electrode chip 320, and impedance was measured in the frequency range of 100 to 10 MHz using a platinum wire as a counter electrode 323 and using an impedance analyzer 324. Then, data in FIGS. 8A and 8B was obtained. FIG. 8A shows an impedance spectrum before the binding of a bacterium, and FIG. 8B shows a change in the impedance due to the binding of a bacterium. It is found that impedance in a region around 1 to 10 MHz has increased with the binding of the bacterium. In this manner, a bacterium can be detected as a change in the alternating-current impedance.

The electrode 302 was connected to a measuring portion that measures the direct current like a potentiostat. A phosphoric acid buffer containing 10 mM potassium ferricyanide was used as a sample solution, and a silver-silver chloride reference electrode that contains saturated KCl as an inner solution and combines the functions of a counter electrode and a reference electrode was used, and then −0.2V was applied to the electrode 302. Then, a redox current decreased by 10% due to the binding of a bacterium. This is because diffusion of potassium ferricyanide, which is a redox substance, was hindered by the bacterium. In this manner, a bacterium can be detected as a change in the redox current. The “current” herein means a rectangular wave of about 1 kHz or lower and a part of such wave.

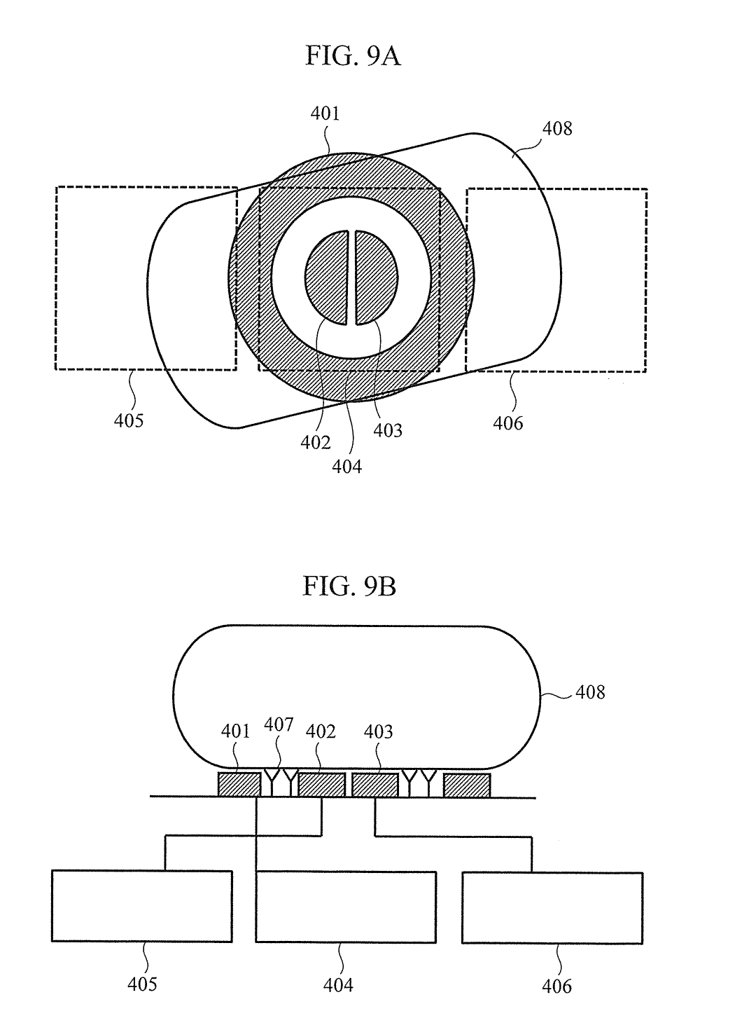

FIG. 9 is a schematic view showing an example of a single partitioned region of a sensor chip in which a sensor that detects the presence or absence of a bacterium and a plurality of sensors that measure metabolites of a substrate co-exist in the single partitioned region. FIG. 9A is a plan schematic view and FIG. 9B is a cross-sectional schematic view. A plurality of sensing portions 402 and 403 of the sensors that measure metabolites of a substrate are arranged such that they are surrounded by a sensing portion 401 of the sensor that detects the presence or absence of a bacterium.

Antibodies 407 to bind to a specific bacterium are immobilized on a region between the sensing portion 401 and the sensing portions 402 and 403. Therefore, when a bacterium 408 is trapped by the antibodies 407, a signal change occurs in a measuring portion 404 connected to the sensing portion 401. For detecting the presence or absence of a bacterium, alternating-current impedance measurement or direct-current redox current measurement can be used. Herein, a substrate is introduced and metabolites of the bacterium are detected with measuring portions 405 and 406 connected to the sensing portions 402 and 403, respectively. For example, a pH sensing film is used as the sensing portion 402 so that a change in pH due to the addition of glucose is detected with the measuring portion 405, and a lactic acid sensing film is used as the sensing film 403 so that lactic acid that is the metabolite of the bacterium is detected with the measuring portion 406. Alternatively, a pH sensing film is used as the sensing portion 402 so that metabolites of the added substrate are detected with the measuring portion 405, and a potassium sensing film is used for the sensing portion 403 so that potassium released as a result the cell membrane being destroyed due to the addition of a surface-active agent or the like is detected with the measuring portion 406. In this manner, when the presence of a bacterium and metabolism of the bacterium or the content of the bacterium are detected in a single partitioned region, it is possible to more accurately determine the type of the bacterium as well as whether the bacterium is live or not.

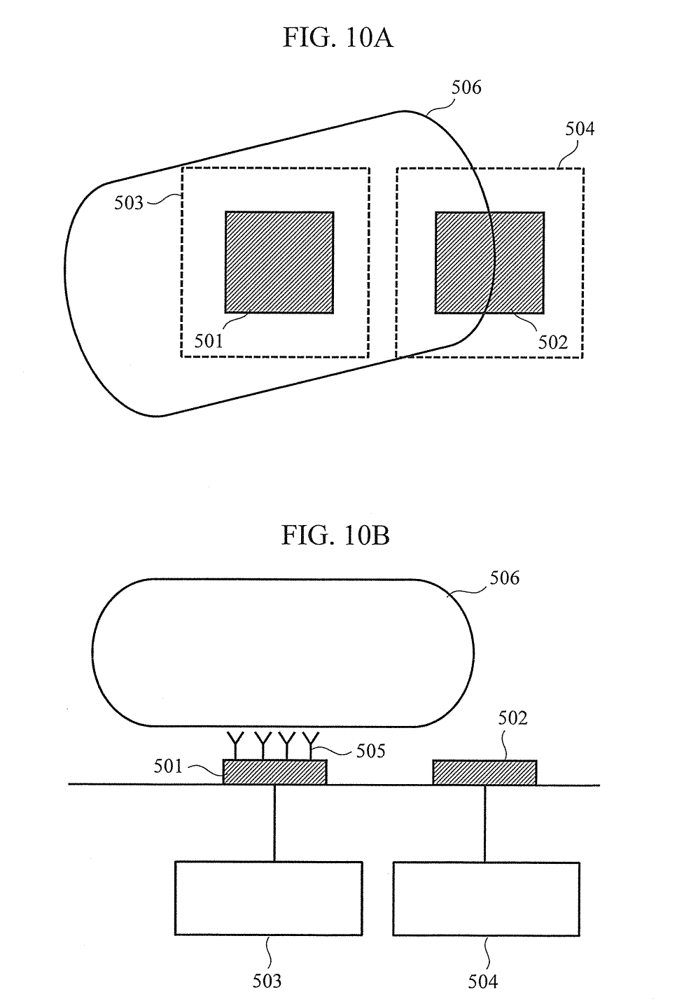

FIGS. 10A and 10B are schematic views showing an example of a single partitioned region of a sensor chip in which a sensor that detects the presence or absence of a bacterium and a sensor that measures metabolites of a substrate co-exist in a single partitioned region. FIG. 10A is a plan schematic view and FIG. 10B is a cross-sectional schematic view. A sensing portion 502 of the sensor that measures metabolites of a substrate is arranged adjacent to a sensing portion 501 of the sensor that detects the presence or absence of a bacterium.

Antibodies 505 to bind to a specific bacterium are immobilized on the sensing portion 501. Therefore, when a bacterium 506 is trapped by the antibodies 505, a signal change occurs in a measuring portion 503 connected to the sensing portion 501. For detecting the presence or absence of a bacterium, alternating-current impedance measurement or direct-current redox current measurement can be used. Herein, a substrate is introduced and metabolites of the bacterium are detected with a measuring portion 504 connected to the sensing portion 502. In this embodiment, the sensing portion 501 of the sensor that detects the presence or absence of a bacterium is located away from the sensing portion 502 of the sensor that measures metabolites of a substrate in comparison with the embodiments shown in FIGS. 4A, 4B, 6A, and 6B, but the sensor chip in this embodiment is still able to detect metabolites.

For the sensing portion, noble metal such as gold or platinum, an oxide film containing carbon, tantalum oxide, or the like, a nitride film containing silicon nitride or the like, an ion sensing film such as a potassium sensing film, or the like can be used. Table 1 shows an example of combinations of sensing portions and measuring portions with respect to substances to be detected.

TABLE 1

| Substance to | | |
|---|---|---|
| be Detected | Sensing Portion | Measuring Portion |
| bacterium, cell, | noble metal, carbon | alternating-current impedance, |
| bead (see JP2011- | | direct-current redox current |
| 232328 A.) | | |
| proton (pH) | tantalum oxide, | potential, FET (field effect |
| | silicon nitride | transistor) |
| electrolyte | ion sensing film, | potential, FET, direct current |
| (Na, K, Mg, Cl) | silver halide (silver | |
| | chloride) | |
| ammonia | ammonia sensing | potential, FET, direct current |
| | film | |

The aforementioned embodiments concern the detection of a bacterium. It is also possible to detect a cell in a similar way by detecting a change in pH, oxygen concentration, carbon dioxide concentration, or the like that results from metabolism when glucose is used as a substrate, using a sensor having a pH sensing portion, an oxygen sensing portion, or a carbon dioxide sensing portion. Accordingly, the state of activity of the cell can be known.

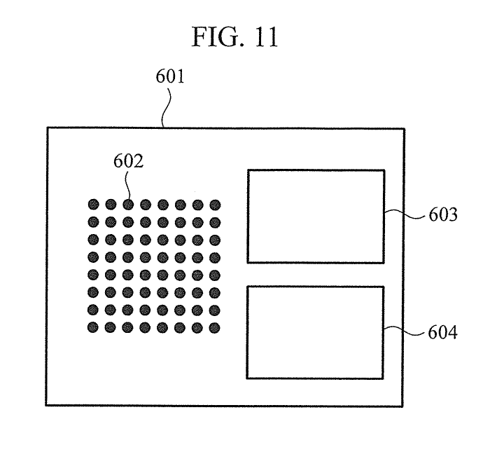

FIG. 11 is a schematic view showing an example of an enzyme sensor array. A sensor chip 601 includes a plurality of partitioned regions 602 indicated by solid black circles. A sensor that detects the presence or absence of a bead, and a sensor that measures an enzyme reaction product generated by an enzyme immobilized on the bead co-exist in a single partitioned region 602. The sensor chip 601 also includes an arithmetic portion 603 and a storage portion 604.

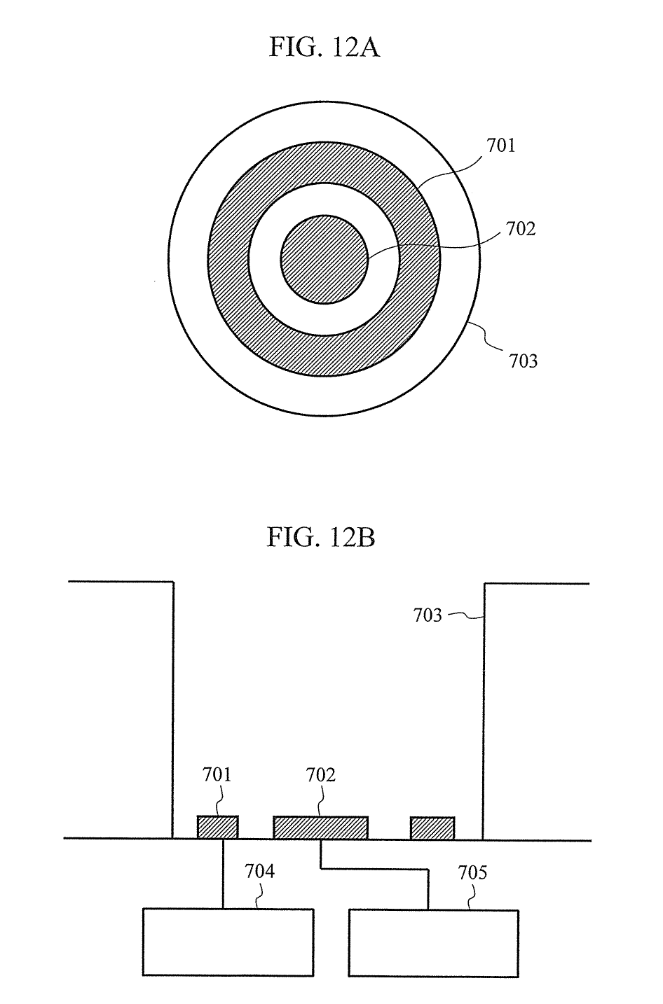

FIGS. 12A and 12B are schematic views of a single partitioned region of the enzyme sensor array. FIG. 12A is a plan schematic view and FIG. 12B is a cross-sectional schematic view. A circular sensing portion 702 of the sensor that measures an enzyme reaction product is arranged such that it is surrounded by a doughnut-shaped sensing portion 701 of the sensor that detects the presence or absence of an enzyme-immobilized bead. Further, these sensing portions are arranged in a well 703 and are surrounded by a wall that is higher than the diameter of the enzyme-immobilized bead to be arranged in the well. The sensing portions 701 and 702 are connected to measuring portions 704 and 704, respectively, in the sensor chip via wires.

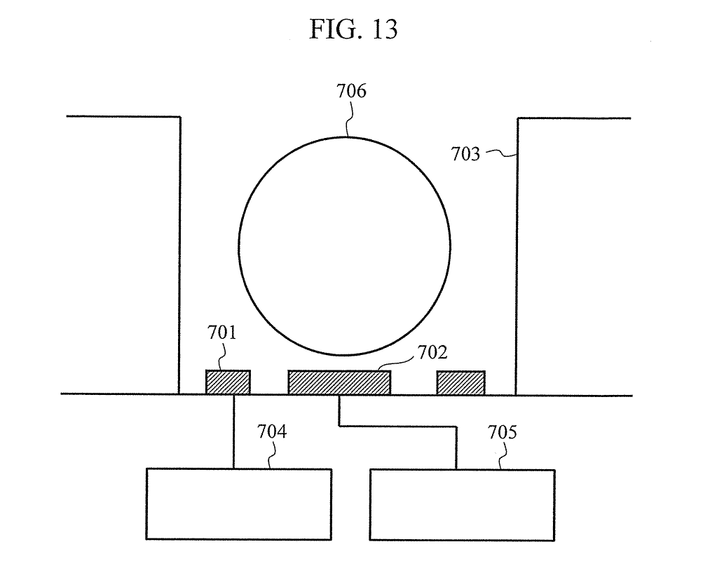

FIG. 13 is a schematic view showing a state in which an enzyme-immobilized bead 706 is arranged in a single partitioned region of the enzyme sensor array. The presence or absence of the bead 706 can be detected by the sensing portion 701 and the measuring portion 704 connected thereto. The size of the bead is about 0.5 to 200 μm. For detecting the presence or absence of a bead, alternating-current impedance measurement or redox current measurement described with reference to FIGS. 5A to 8B can be used. When a mixture of an analyte and a substrate is introduced in the state of FIG. 13 in which the bead is arranged in the well 703, an enzyme reaction occurs due to the enzyme that modifies the bead 706. A reaction product of the enzyme is measured by the sensing portion 702 and the measuring portion 705 connected thereto.

When an antibody-immobilized bead is used instead of the enzyme-immobilized bead 706 in a similar arrangement to that in FIG. 13, an immunosensor can be provided. The principle of the measurement is similar to the Enzyme-Linked ImmunoSorbent Assay (ELISA). First, an analyte is introduced so that a substance to be measured in the analyte is made to bind to the antibody immobilized on the bead. Next, an enzyme-labeled secondary antibody is introduced to obtain a bonding state of the enzyme—the substance to be measured—the secondary antibody. Further, a substrate is introduced so that it reacts with the enzyme labeled on the secondary antibody, whereby a reaction product is obtained. The quantity of the reaction product is measured with the sensing portion 702 and the measuring portion 705 connected thereto, so that the concentration of the substance to be measured in the analyte is determined. For the enzyme labeled on the secondary antibody, glucose oxidase or alkaline phosphatase can be used, for example. For the substrate, glucose, aminophenol phosphate, or ascorbic acid-2-phosphate esters can be used, for example. For the detection scheme, a redox current scheme or a redox potential scheme can be used, for example.

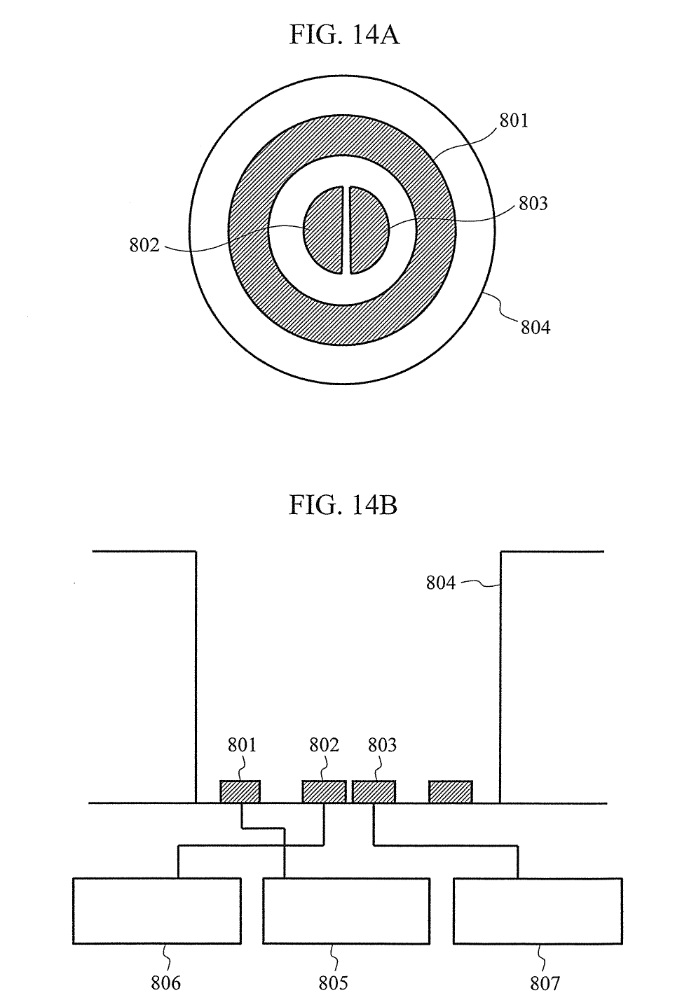

FIGS. 14A and 14B are schematic views showing another example of a single partitioned region of an enzyme sensor array. FIG. 14A is a plan schematic view and FIG. 14B is a cross-sectional schematic view. A plurality of sensing portions 802 and 803 of sensors that measure an enzyme reaction product are arranged such that they are surrounded by a doughnut-shaped sensing portion 801 of a sensor that detects the presence or absence of an enzyme-immobilized bead. Further, the sensing portions 801 to 803 are arranged in a well 804, and are surrounded by a wall that is higher than the diameter of the enzyme-immobilized bead to be arranged in the well. The sensing portions 801, 802, and 803 are connected to measuring portions 805, 806, and 807, respectively, in the sensor chip via wires.

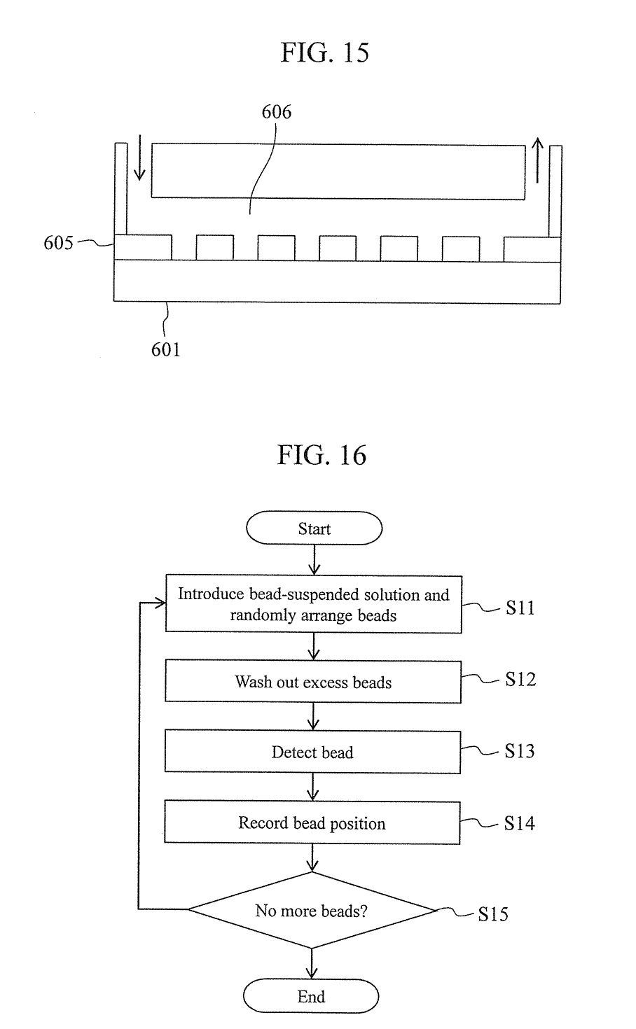

FIG. 15 is a schematic view of a measurement chip that uses the sensor chip in FIG. 11. A flow channel 606 is further formed above a well layer 605 formed on the sensor chip 601. In the drawing, a solution inlet port is arranged on the left side, and a solution outlet port is arranged on the right side. When an enzyme-immobilized bead is arranged on each well of the measurement chip, the measurement chip can function as an enzyme sensor array.

Next, the procedures for arranging an enzyme-immobilized bead in each well of the sensor chip will be described. FIG. 16 shows the flow. First, in step 11, a solution in which enzyme-immobilized beads having given enzymes A immobilized thereon are suspended is introduced from the solution inlet port. The enzyme-immobilized beads are randomly arranged in the plurality of wells of the sensor chip 601 through diffusion or convection of the solution or through centrifugal force according to circumstances. Next, in step S12, excess enzyme-immobilized beads that have not entered the wells are washed away. Then, a solution suitable for detecting the beads is introduced through the flow channel to inspect a well in which an enzyme-immobilized bead has been introduced, using the sensor that detects the presence or absence of a bead in step 13. The presence or absence of a bead is determined by the arithmetic portion 603. A well in which the presence of a bead is detected contains introduced therein the enzyme-immobilized bead having the enzyme A immobilized thereon. Thus, in step S14, the enzyme A and the position of the well in which the presence of the bead is detected are recorded in association with each other. Such information may be recorded in another recording medium or nonvolatile memory incorporated in the sensor chip (FIG. 11, 604).

Next, through the determination in step 15, the operations in S11 to S14 are repeated for enzyme-immobilized beads having enzymes B of a different type immobilized thereon. At this time, a well in which the presence of a bead is newly detected in the operation in step S13 contains introduced therein the enzyme-immobilized bead having the enzyme B immobilized thereon. Thus, in step S14, the enzyme B and the position of the well in which the bead is newly detected this time are recorded in association with each other. Similar operations are performed on all of the enzyme-immobilized beads having immobilized thereon enzymes C, D, . . . of different types. Consequently, information about the positions of wells and the types of enzyme-immobilized beads arranged in the wells can be obtained. When a plurality of types of sensing portions of sensors are located in a single partitioned region as shown in FIG. 14, the range of the types of enzyme-immobilized beads that can be applied will increase.

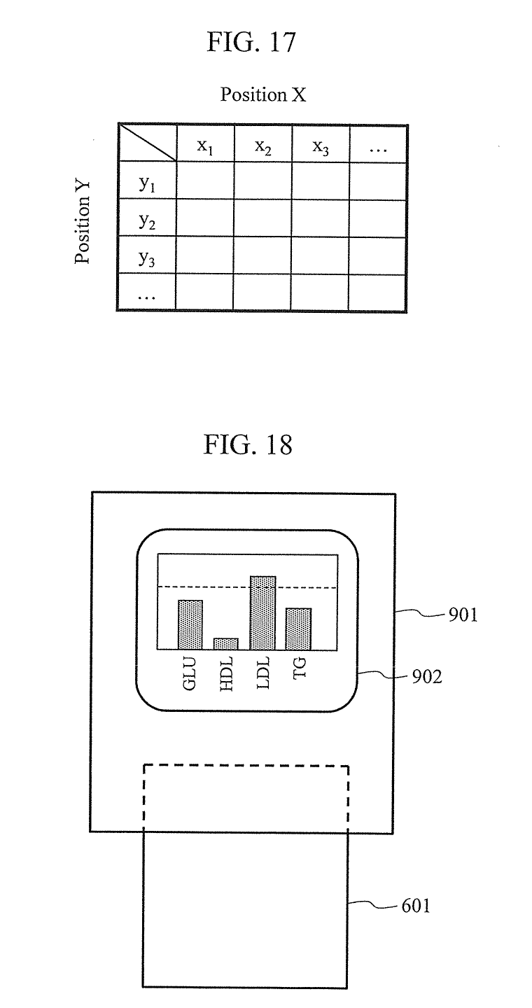

FIG. 17 is a diagram showing the correspondence between the positions of wells on the sensor chip 601 and the types of enzyme-immobilized beads arranged in the respective wells. The position X and the position Y are information to identify the position of each partitioned region 602 arranged on the sensor chip 601 in a two-dimensional array. For example, when a bead having an enzyme A immobilized thereon is detected at a position (xₘ,yₙ), the position of the well and the type of the enzyme are associated with each other such that (xₘ,yₙ)=A. Information indicating the positions of wells that contain introduced therein enzyme-immobilized beads having all of the prepared types of enzymes A, B, C, . . . immobilized thereon is acquired and stored in this manner. When information is to be stored in the storage portion 604 incorporated in the sensor chip 601, information about the types of enzymes is input from an input device connected to the sensor chip 601. Meanwhile, when information is to be stored in an external storage portion, for example, a storage portion of a measuring device 901 described below with reference to FIG. 18, after an enzyme-immobilized bead having one type of enzyme immobilized thereon is introduced, operations of acquiring position information on a well containing the bead introduced therein from the sensor chip 601, and storing information on the introduced enzyme into a storage medium are repeated.

FIG. 18 is a schematic view of a measuring device on which the sensor chip is set. As shown in FIG. 18, the sensor chip 601 having the obtained enzyme-immobilized beads introduced therein is set on the measuring device 901 so that a plurality of items can be measured concurrently. Data transfer between the sensor chip 601 and the measuring device 901 may be performed through, by providing a terminal on each of the sensor chip 601 and the measuring device 901, mechanical contact between the terminals or through noncontact communication means.

For example, when the blood components are measured, a mixed solution of blood serum and a substrate solution is introduced through the flow channel 606 shown in FIG. 15. When the blood serum contains a component corresponding to the substrate, only the blood serum may be introduced. Consequently, an enzyme reaction corresponding to each enzyme-immobilized bead occurs in each well, producing a reaction product from the substrate. Each measuring portion of the sensor chip 601 measures the product using a sensor for measuring a product, which is different from a sensor for detecting a bead, arranged in the well. The measuring device 901 can, by checking the measured value obtained in each well against the information about the type of the enzyme-immobilized bead arranged in each well recorded in advance (FIG. 17), associate the measured value of each well with the measurement item, and can concurrently measure a plurality of measurement items. When a plurality of measured values are obtained with regard to a single measurement item, a statistical process such as determination of the arithmetic mean may be performed to determine the final measured value. The measurement result is displayed on a display potion 902. The correspondence between wells and beads may be recorded in the sensor chip or be obtained by referring to data in a remote location on the basis of the ID of the sensor chip. FIG. 18 shows glucose (GLU), cholesterol (HDL, LDL), and neutral fat (TG) together with the reference values (dotted line). The reference value of each measurement item is stored in the measuring device 901. In the case of the display example shown, the reference values of all measurement items are displayed such that they are at equal level, and the measured value of each measurement item is displayed with a bar chart that is proportionally expanded or shrunk with respect to the reference value.

Instead of the enzyme-immobilized beads, it is also possible to use antibody-immobilized beads. In that case, a measurement chip on which antibody-immobilized beads are arranged is obtained through the flow shown in FIG. 16. An analyte (e.g., blood, a body fluid, a food extract, or a soil extract) is introduced through the flow channel of the measurement chip, and the analyte is washed away after the passage of a time (typically, 10 minutes to 1 hour) that is necessary for an antigen-antibody reaction. Then, an antibody as a label is further made to react with a substance to be measured in the analyte that has been trapped on the antibodies on the bead. After washing, a solution containing a substrate to react with the antibody as the label is introduced. Consequently, a reaction occurs in which a product is generated in each well. The product is measured with a measuring portion using a sensor for measuring a product that is different from a sensor for detecting a bead. The measuring device 901 can, by checking the measured value obtained in each well against the information about the type of the antibody-immobilized bead arranged in each well registered in advance, concurrently measure a plurality of items.

Table 2 shows an example of combinations of measurement items, enzymes used for the enzyme-immobilized beads, and detection schemes. As a redox potential sensor, a sensor such as the one described in JP2008-128803A can be used, for example.

TABLE 2

| Measurement | | |
|---|---|---|
| Item | Enzyme | Detection Scheme |
| glucose | glucose oxidase | redox current sensor |
| | | redox potential sensor |
| glucose | hexokinase, glucose-6- | redox current sensor |
| | phosphate dehydrogenase, | redox potential sensor |
| | diaphorase | |
| cholesterol | cholesterol esterase, | redox current sensor |
| | cholesterol dehydrogenase, | redox potential sensor |
| | diaphorase | |
| urea | urease | pH sensor, ammonium |
| | | sensor |

It should be noted that the present invention is not limited to the aforementioned embodiments, and includes various variations. For example, although the aforementioned embodiments have been described in detail to clearly illustrate the present invention, the present invention need not include all of the structures described in the embodiments. It is possible to replace a part of a structure of an embodiment with a structure of another embodiment. In addition, it is also possible to add, to a structure of an embodiment, a structure of another embodiment. Further, it is also possible to, for a part of a structure of each embodiment, add/remove/substitute another structure.

### REFERENCE SIGNS LIST

101, 601: Sensor chips
102, 602: Partitioned regions
201, 202, 401, 402, 403, 501, 502, 701, 702, 801, 802, 803: Sensing portions
203, 204, 404, 405, 406, 503, 504, 704, 705, 805, 806, 807: Measuring portions
205, 304, 407, 505: Antibodies
302: Electrode
303: Wire
210, 310, 408, 506: Bacteria
320: Electrode chip
321: Cell
322: Solution to be Measured
323: Counter electrode
324: Impedance analyzer
603: Arithmetic portion
604: Storage portion
703, 804: Wells
706: Enzyme-immobilized bead
605: Well layer
606: Flow channel
901: Measuring device
902: Display portion

What is claimed is:

1. A sensor chip comprising:
a board;
a plurality of partitioned regions provided on a surface of the board;
a plurality of types of sensing portions provided in each of the plurality of partitioned regions; and
a plurality of detection portions connected to the respective sensing portions.

2. The sensor chip according to claim 1, wherein each partitioned region has about the same size as a target to be measured.

3. The sensor chip according to claim 2, wherein the target to be measured is a cell or a bacterium.

4. The sensor chip according to claim 2, wherein one of the plurality of sensing portions is connected to a detection portion that detects the presence or absence of the target to be measured.

5. The sensor chip according to claim 1, wherein each partitioned region is a well provided on the board.

6. The sensor chip according to claim 1, wherein the plurality of sensing portions are arranged such that one of the sensing portions surrounds another sensing portion.

7. The sensor chip according to claim 1, wherein the plurality of sensing portions have different shapes.

8. The sensor chip according to claim 1, wherein each sensing portion includes one of noble metal, carbon, an oxide film, a nitride film, or an ion sensing film.

9. The sensor chip according to claim 1, wherein each detection portion is an alternating-current impedance measuring portion, a direct-current redox current measuring portion, or a potential measuring portion.

10. The sensor chip according to claim 3, wherein each partitioned region is at least partially modified by an antibody for trapping the target to be measured.

11. A measurement method comprising:
introducing a solution containing a target to be measured onto a sensor chip, the sensor chip having a board and a plurality of partitioned regions provided on a surface of the board, and each partitioned region having a first sensing portion for detecting the presence or absence of the target to be measured and a second sensing portion for measuring a metabolite of a substrate;
detecting, with the first sensing portion, the presence or absence of the target to be measured in each partitioned region;
introducing a substrate onto the sensor chip; and
measuring, with the second sensing portion, a metabolite of the substrate generated by the target to be measured.

12. The measurement method according to claim 11, wherein the target to be measured is a cell or a bacterium, and each partitioned region is at least partially modified by an antibody for trapping the target to be measured.

13. A measurement method comprising:
introducing a solution containing first beads having first enzymes immobilized thereon onto a sensor chip, the sensor chip having a board and a plurality of wells formed on a surface of the board, and each well having a first sensing portion for detecting the presence or absence of the bead and a second sensing portion for measuring a metabolite of a substrate;
detecting, with the first sensing portion of each well, the presence or absence of one of the first beads;
removing excess first beads that have not entered the well;
storing a well in which the first bead is detected and the first enzyme in association with each other;
introducing a solution containing second beads having second enzymes immobilized thereon onto the sensor chip;
removing excess second beads that have not entered the well;
detecting, with the first sensing portion, the presence or absence of one of the second beads in each well excluding the well in which the first bead is detected;
storing the well in which the second bead is detected and the second enzyme in association with each other;
introducing a mixture of an analyte and a substrate onto the sensor chip;
measuring, with the second sensing portion, a reaction product of the first enzyme or the second enzyme; and
obtaining information about a measurement item measured with the second sensing portion with reference to the stored correspondence between the well and the first enzyme or the second enzyme.

14. A measurement method comprising:
introducing a solution containing first beads having first enzymes immobilized thereon onto a sensor chip, the sensor chip having a board and a plurality of wells formed on a surface of the board, and each well having a first sensing portion for detecting the presence or absence of the bead and a second sensing portion for measuring a metabolite of a substrate;
detecting, with the first sensing portion of each well, the presence or absence of one of the first beads;
removing excess first beads that have not entered the well;
storing a well in which the first bead is detected and the first enzyme in association with each other;
introducing a solution containing second beads having second enzymes immobilized thereon onto the sensor chip;
removing excess second beads that have not entered the well;
detecting, with the first sensing portion, the presence or absence of one of the second beads in each well excluding the well in which the first bead is detected;
storing the well in which the second bead is detected and the second enzyme in association with each other;
introducing an analyte containing a substance to be measured onto the sensor chip, and causing the substance to be measured to be trapped by the first enzyme immobilized on the first bead or the second enzyme immobilized on the second bead;
causing the substance to be measured trapped by the first enzyme or the second enzyme to react with an enzyme-labeled secondary antibody;
introducing a solution containing a substrate to react with the enzyme labeled on the secondary antibody;
measuring, with the second sensing portion, a reaction product of the enzyme; and
obtaining information about a measurement item measured with the second sensing portion with reference to the stored correspondence between the stored well and the first antibody or the second antibody.

15. The measurement method according to claim 13, wherein the first sensing portion measures an alternating-current impedance or a direct-current redox current.

16. The measurement method according to claim 14, wherein the first sensing portion measures an alternating-current impedance or a direct-current redox current.
